(19) United States
(12) Patent Application Publication
(10) Pub. No.: US 2012/0088212 A1
(43) Pub. Date: Apr. 12, 2012

(54) COMPUTERIZED SYSTEM FOR ADDICTION CONTROL ESPECIALLY CALORIE, DIET AND WEIGHT CONTROL

(76) Inventor: Tzachi KNAAN, Ramat Gan (IL)

(21) Appl. No.: 13/270,473

(22) Filed: Oct. 11, 2011

Related U.S. Application Data
(60) Provisional application No. 61/392,499, filed on Oct. 13, 2010.

(30) Foreign Application Priority Data
Oct. 11, 2010 (IL) 208629

(51) Int. Cl. G09B 19/00

(52) U.S. Cl. 434/236

(57) ABSTRACT

A computerized device and system providing a subject with a behavioral plan for maintaining or improving wellness in the field of addiction, especially eating and weight control is disclosed, comprising a computer usable medium with computer readable program code embodied, a means of inputting data concerned with psychological and physical descriptions of the subject's behavior into a retrievable and updatable database. A processing means with computer programmable code for processing input data to produce output data and a display means for displaying information is provided. The computer programmable code is adapted for calculating the optimal eating behavior to be prompted to the subject based upon application of an algorithm that processes input data and outputs a time resolved behavioral plan.

### BACKGROUND OF THE INVENTION

1. Field of the Invention

The present invention relates to a computerized system capable of processing and interpreting information input in order to output warnings or recommendations as well as reporting historical behavioral patterns in order to modify addictive behavior, especially in relation to weight control.

2. Background Art

Obesity is a widespread problem in the developed world, where food is available in excess and large sections of the population live mostly sedentary lives. Obesity has an enormous influence on health and is a risk factor in many life-limiting conditions such as heart disease, stroke, cancer and diabetes among others, as well as severely affecting fertility in women. Moreover obesity and excess weight in general has a substantial psychological effect, impacting on a person's body image and mental world. Weight gain is usually a consequence of intake of surplus calories from food combined with lack of physical activity, this is in large part to the fact that many people are not aware of their eating patterns and eat over the biological requirement of their bodies, without any reference to their true sense of hunger and satiation.

It would therefore be useful for an overweight or obese person to have a means of losing weight by self-treatment which incorporates and instructs the user to recognize and characterize as well as learn their true sense of satiation and hunger, especially in a way that is easily accessible whenever the user requires it, as for example in a computerized system into which the user can feed information and receive immediate feedback. Moreover such a tool could be used both by those loosing weight independently and also for health care professionals such as dietitians, doctors, psychologists and others with their patients as a combined diagnostic and treatment tool.

U.S. Pat. No. 6,932,766 discloses a satiety meter and relates to a computerized handheld pocket-sized device and method for registering satiety and satiety response useful for aiding a person, in a controlled manner, to appreciate when to eat. The device of U.S. Pat. No. 6,932,766 comprises in-/output means, electronic memory, a display screen and a modified Borg type scale for rating of satiety.

Banos et al., (2009) describe an electronic Personal Digital Assistant (PDA) system for recording food and physical activity for the treatment of childhood obesity. The PDA application described is one aspect of a larger e-health platform, an intelligent e-therapy (eTI) system known as ETIOBE, for the treatment of obesity. The system includes various communication and information applications for transferring, managing and storing information and for interpreting and reacting to it and is personalized and adapted in accordance with the patient's answers and characteristics. ETIOBE comprises a clinical support system that allows clinical intervention through the internet, a home support system where the child or user can find the tasks the clinician has selected and a mobile support system that allows the user to self-monitor and register dietary and physical activity. Thus the ETIOBE system acts as a self-registration system accessible to clinicians who in turn provide feedback according to the users' self-registration input.

While a variety of computerized diet and addiction control methods and devices are available to consumers, there is a long-felt need for a device and method that incorporates all aspects of satiety and food intake habits and addiction habits from the user in a holistic manner in order to instruct and guide the user in losing weight or controlling addictions in a simple and quick input and response system independent of other clinical intervention.

### SUMMARY OF THE INVENTION

The present invention relates to the field of computerized devices and systems for monitoring and modifying addictive behaviors and in particular those related with eating and weight loss.

It is therefore one object of the present invention to disclose a device for providing a subject with a behavioral plan for maintaining or improving wellness comprising a computer usable medium having a computer readable program code embodied therein, said computer usable medium comprising; a means of inputting data into a retrievable and updatable database, said input data concerned with food intake data, nutritional value data, satiety data, lifestyle data, emotional state or environment data, physical state or environment data, genetic data, general health data, BMI data, weight data, temporal data, geographical data, general habit data, smoking data, prescribed drug data, alcohol intake data, self medication data, non prescribed drug data, leisure activity data, hobby data, personal relationship data, fitness data, employment data and social data or any combination thereof; a retrievable and updatable database comprising said data; a processing means comprising a computer programmable code for processing said input data to produce output data; a display means for displaying information concerned with said input and output data wherein said computer programmable code is adapted for calculating the optimal eating behavior to be prompted to said subject based upon application of an algorithm, said algorithm provided for processing said input data and outputting a time resolved behavioral plan

It is yet another object of the present invention to disclose a device in which the behavioral plan is concerned with diet and eating.

In another aspect of the present invention a device is disclosed, incorporating a numerical scale for counting calorific intake which in conjunction with a computer implemented system and device is useful for weight control.

It is yet another object of the present invention to disclose a device in which daily calorie count is quantified according to calorie range categories, rather than individual foods.

In a preferred embodiment of the present invention a device is disclosed, incorporating a numerical scale for characterization of the reason for eating—“the mental environment”, for example boredom, sadness, joy, hunger or any other emotion associated with eating.

In a preferred embodiment of the present invention a device is disclosed, incorporating a numerical scale for characterization of the physical environment for eating, for example eating while eating alone, in company, in front of the TV or while carrying out a different activity, eating at home or in a restaurant.

In yet another preferred embodiment of the present invention a device is disclosed, said device is adapted to process input data consisting of a numerical rating of between 1 to 5 for hunger combined with a verbal description immediately prior to a meal or food intake.

In yet another preferred embodiment of the present invention a device is disclosed, said device is adapted to process input data consisting of a numerical rating of emotion combined with a verbal description immediately prior to a meal or food intake.

It is yet another object of the present invention to disclose a device adapted to process input data consisting of a rating of between 1 to 5 for satiation combined with a verbal description immediately following a meal or food intake.

It is yet another object of the present invention to disclose a device adapted to process input data consisting of a rating of between 1 to 5 for calorie categories immediately following a meal or food intake.

It is yet another object of the present invention to disclose a device adapted to process input data consisting of a visual rating of eating position during a meal or food intake.

It is yet another object of the present invention to disclose a device adapted to process input data consisting of a numerical rating of between 1 to 5 for the level of concentration on eating and physical environment during a meal or food intake.

It is yet another object of the present invention to disclose a device in which the A1 dichotomy grading for hunger immediately prior to a meal or food intake is scored according to TABLE 1.

It is yet another object of the present invention to disclose a device in which the A2 dichotomy grading for emotional and psychological reasons for eating immediately prior to a meal or food intake is scored according to TABLE 2.

It is yet another object of the present invention to disclose a device in which the level of satiation immediately following a meal or food intake is scored according to TABLE 3.

It is yet another object of the present invention to disclose a device in which calorie intake during a meal or food intake is scored according to TABLE 4.

It is yet another object of the present invention to disclose a device in which the B1 dichotomy grading of eating position is scored according to TABLE 5.

It is yet another object of the present invention to disclose a device in which the B2 dichotomy grading of eating environment and concentration during a meal is scored according to TABLE 6.

It is yet another preferred embodiment of the present invention to disclose a device in which the behavioral plan is concerned with types of addiction.

It is yet another preferred embodiment of the present invention to disclose a behavioral plan that is concerned with eating disorders such as bulimia or anorexia

It is yet another object of the present invention to disclose a device in which the behavioral plan is for an addiction selected from the group consisting of alcoholism, non-prescription drug addiction, prescription drug addiction, sex addiction, smoking, computer gaming addiction, gambling addiction, and any other forms of addiction.

It is yet another object of the present invention to disclose a device adapted for use in therapeutic programs.

It is yet another object of the present invention to disclose a device adapted for use in conjunction with drug therapy.

It is yet another object of the present invention to disclose a device adapted for use in conjunction with psychological or psychiatric therapy.

It is one object of the present invention to disclose a system for providing a subject with a behavioral plan for maintaining or improving wellness comprising; a computer usable medium having a computer readable program code embodied therein, said computer usable medium comprising; a means of inputting data into a retrievable and updatable database, said input data concerned with food intake data, nutritional value data, satiety data, lifestyle data, emotional state or environment data, physical state or environment data, genetic data, general health data, BMI data, weight data, temporal data, geographical data, general habit data, smoking data, prescribed drug data, alcohol intake data, self medication data, non prescribed drug data, leisure activity data, hobby data, personal relationship data, fitness data, employment data and social data or any combination thereof; a retrievable and updatable database comprising said data optionally residing on a server; a processing means comprising a computer programmable code for processing said input data to produce output data; a display means for displaying information concerned with said input and output data wherein said computer programmable code is adapted for calculating the optimal eating behavior to be prompted to said subject based upon application of an algorithm, said algorithm provided for processing said input data and outputting a time resolved behavioral plan.

### BRIEF DESCRIPTION OF THE DRAWINGS

The invention is herein described, by way of example only, with reference to the accompanying drawings. The particulars shown are by way of example only and for the purposes of illustrative discussion of the preferred embodiments of the present invention only. The description taken with the drawings should make apparent to those skilled in the art how several forms of the invention may be embodied in practice.

In the drawings:

FIG. 1 is a schematic illustration of the flow of input and output in the computerized system of the present invention; and

FIG. 2 is a flow diagram illustrating the input of information into the computerized system of the present invention; and

FIG. 3 is a pictorial illustration of the TAB options available to users of the computerized system of the present invention; and

FIG. 4 is a schematic illustration of eating events as described by the computerized system of the present invention; and

FIG. 5 is a schematic illustration of the scoring options in the computerized system of the present invention; and

FIG. 6 is a pictorial illustration of a screen shot representing the start page of the computerized weight loss system; and

FIG. 7 is a pictorial illustration of a screen shot of the calorie category page of the computerized weight loss system of the present invention.

### DETAILED DESCRIPTION OF THE PREFERRED EMBODIMENTS

The present invention solves many of the problems of current methods and systems of weight loss and control by providing a system and method of instructing a consumer on how to loose weight by processing and interpreting a range of variables associated with satiety and eating habits or for modifying behavioral patterns to improve wellness for any other behavior or addiction.

The term “user interface” hereinafter refers to any computerized device, such as computer, mobile phone, personal organizer, PDA (personal digital assistant) or digital television, cable television or any other computerized device capable of receiving, processing and transmitting inputted information and delivering an output.

The term “screen” hereinafter refers to the visual display process presented to a user by the computer interface.

The term “BMI” hereinafter refers to Body Mass Index, a measure of weight relative to height. The BMI is taken as a measure of the weight (in kgs) of a subject divided by the height (in meters) squared, thus;

BMI=x kg÷(y m)²

For the purposes of this invention, a healthy BMI is considered to in the range of 20-25, a BMI of more than 25 is considered as overweight and a BMI of more than 30 is considered obese. It is well within the scope of the present invention wherein the BMI of an overweight or obese human subject may be reduced by continuous use of the device.

Conversely, a BMI of less than 20 is considered to be underweight.

It is therefore a preferred embodiment of the present invention to provide a system and method of assisting an overweight or obese consumer, or any person desiring an improvement in their eating habits. In this embodiment a computerized system interprets a set of personal data inputs and reacts according to the input to regulate and instruct the consumer in healthy eating practices. The system is able to monitor and interpret data related to the user's eating habits, 24 hours a day over a prolonged period of time. Thus for example in this embodiment the following input data is submitted by the user;
i. Rating of between 1 to 5 for satiation and hunger immediately prior to and after a meal or food intake,
ii. Daily calorie count according to meal type categories, rather than individual foods
iii. Characterization of the reason for eating—“the mental environment”, for example boredom, sadness, joy, hunger or any other emotion associated with eating
iv. Characterization of the physical environment for eating, for example eating while seated, standing up, eating alone, in company, in front of the TV or while carrying out a different activity, eating at home or in a restaurant.

It is a further embodiment of the present invention to provide output data to the user in reaction to input data received from the user. In this embodiment the output data includes but is not limited to the following output options;
i. Recommendations immediately reacting to the user's input.
ii. PUSH messages throughout the day according to the nature of behavior
iii. Graphic and numerical description of the emotional, behavioral, and calorie connotations in a daily/per date/ monthly summary.
iv. Behavioral change trends over the use of the application
v. Graphic description of change in body weight/size (various measurements etc.) as a result of the treatment.

Thus in this embodiment the user receives instructive output that enables the user to regulate their eating patterns and to understand and control adverse eating patterns so as to better control calorie intake. Also in this embodiment the user receives output containing quantitative data relating to the user's calorie intake and changes in their weight and size.

In another preferred embodiment of the present invention the novel system of the present invention will use computerized ability: backup, input and output of internal memory (local database data) and external memory (server). In addition in this embodiment a website acting as a “mirror image” for all the processes occurring within the application is provided, other systems such as cable television systems or mobile phones or any other computerized system with internet access may also provide the user with access to the application. Furthermore the cellular software and application is presented in at least three different languages.

In a preferred embodiment a computerized weight control system would be available to users by the following means;
i. For payment by end user
ii. As a complementary product to promote foods in the field of nutrition and weight loss and for payment by the companies wishing to market these items. For example a code for entering the novel system would be available free to those purchasing a specific brand of cereals.
iii. By selling the application to international food companies or those dealing with sports to characterize them as health promoters.
iv. As a special purchase by organizations for their employees or for their insured customers, in national or international health clinic organizations or health insurance companies
v. As a downloadable b-free version in the sponsored by a commercial company.

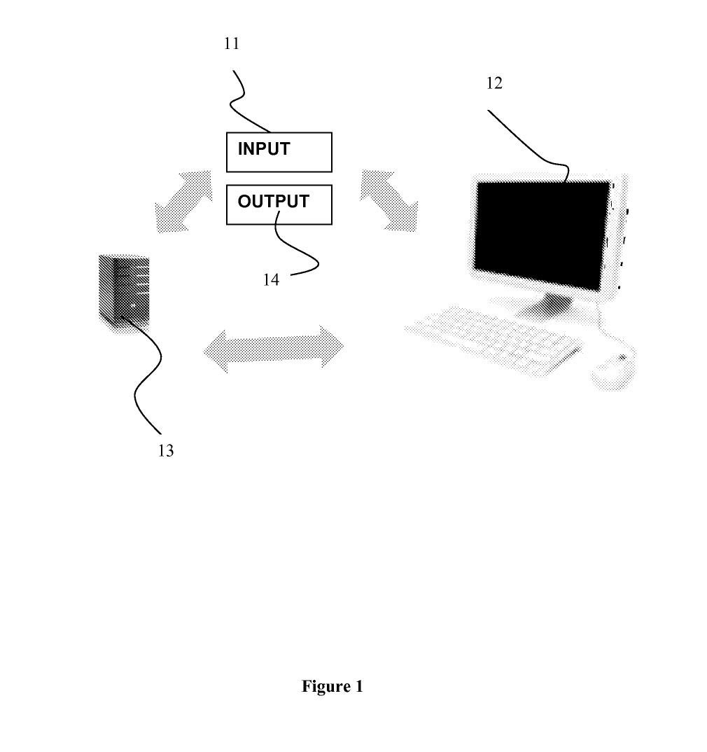

Reference is now made to FIG. 1 in the drawings showing a flow diagram representing the flow of information between the computerized system and the user, in which a user submits input 11 information through the computerized device that is the user interface 12 which is then processed through a server 13. Once the server has processed the information in the application running through the server, it feeds an output 14 through the user interface in reaction to the input data received.

In a further preferred embodiment of the present invention, output information may be presented to the user through the user interface and processed through two possible channels. In this embodiment push services such as reminders and recommendations specific to the user or more general messages for all users are processed and directed through a server. Teasers such as immediate tips are processed through the local database of the computerized user interface. Also in this embodiment teasers and push messages are additionally generated by human operators that have authorized access to the user's input and output, as in for example dieticians on call 24 hours a day to further process the input information. Thus in this embodiment the output including teasers and push messages are provided either solely by the computer system or solely by human input or by both human and computerized processing.

In a further preferred embodiment of the present invention, the behavioral weight loss computerized system may be adapted so as to treat and modify behaviour for any addictive behavior, for example: alcoholism, non-prescription drug addiction, prescription drug addiction, sex addiction, smoking, computer gaming addiction and gambling addiction or other eating disorders such as bulimia or anorexia nervosa.

In a further preferred embodiment the computerized eating behaviour and weight loss system and adapted addictive behavior systems are integrated as a part of a therapeutic programme and/or in conjunction with drug therapy or pyschological or psychiatric therapy

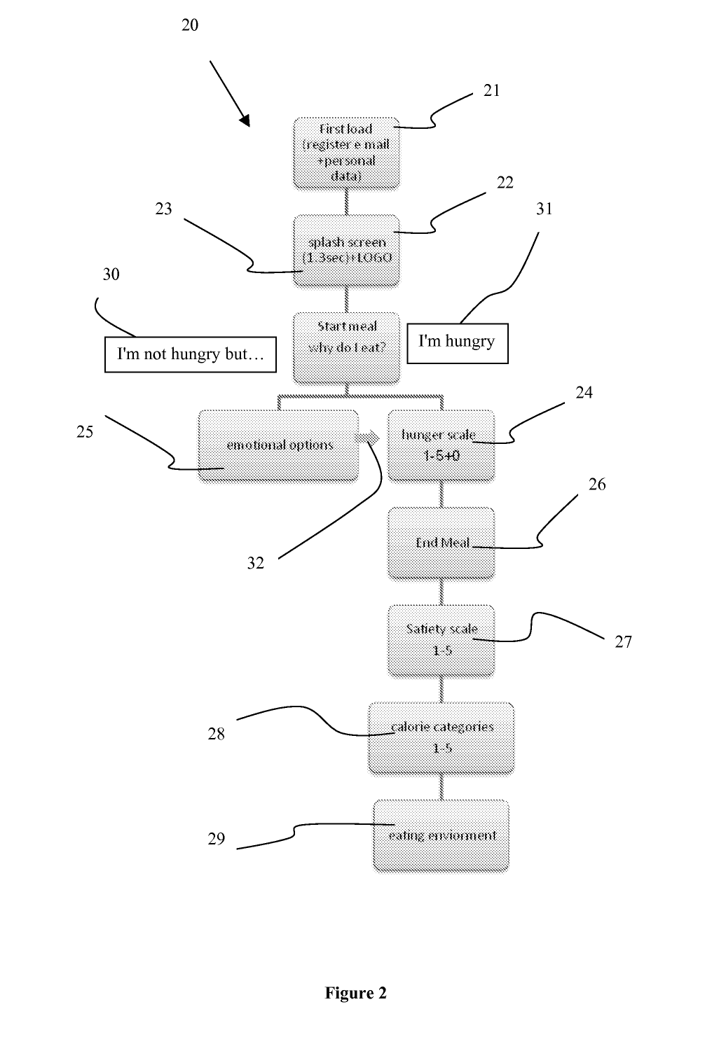

In FIG. 2, an example screen shot flow diagram 20 is shown for the novel weight loss system and method of the present invention. In this preferred embodiment, different screen options that may be presented sequentially to the user through the user interface are shown. In the first stage of entry 21 to the novel application, the user is presented with a screen requesting identifying information for a first load or login. In this first stage the user may register an email address and/ or personal data. A subsequent “splash screen” 22 is presented briefly, for about 1-2 seconds in which the user is not required to input information. In this “splash screen” stage the user is presented with the logo of the application or other visual information until the application has fully loaded. In a subsequent a screen for the start of a meal is presented 23, in which the user is asked for their reason for eating. In this screen two possible options are presented as reasons for starting a meal or eating. One screen represents the emotional or psychological options for eating 25, this option is presented when the user is not hungry 30 but is eating for emotional reasons, thus in this screen the user may input information regarding their emotional or psychological state, eg bored, depressed, happy or any other relevant emotional state. The other screen option is selected when the user is eating because they are hungry 31, in this screen the user's physical condition at the beginning of the meal is assessed through a hunger scale 24. In addition users that are not hungry but are eating for emotional reasons are then redirected 32 to the hunger scale screen 24 from the emotional options screen. In the subsequent screen the user inputs data at the end of the meal 26. Subsequent screens allow the user to input information regarding the meal, such as satiety 27 according to a satiety scale of 1 to 5, calorie categories 28 assessed on a scale of 1 to 5 and the eating environment 29, such as standing up, in front of the television, at home, in a restaurant or any other eating environment. Thus in this embodiment the user is presented with different screens to input information to allow the novel weight loss system to react and give instructive output.

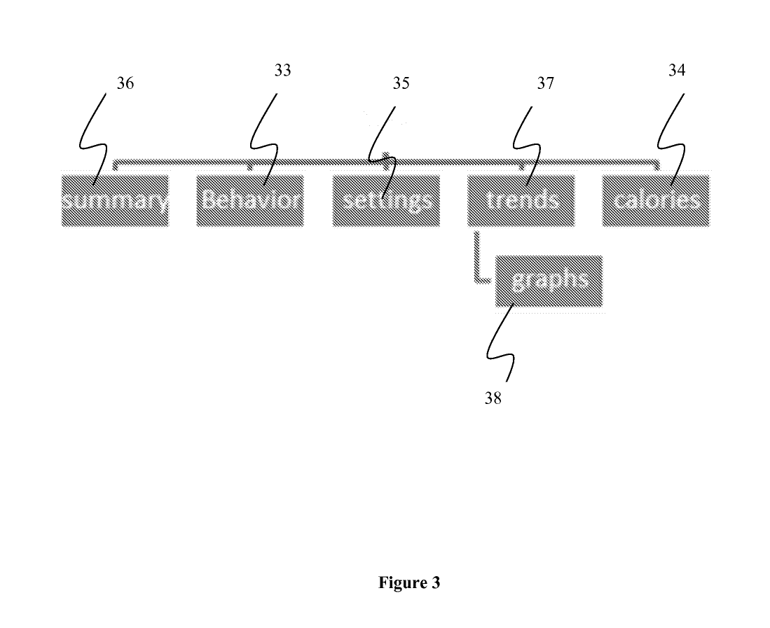

Reference is now made to FIG. 3 in the drawings in which a variety of tabs that may be presented to the user through the user interface in order to select different output categories is presented. Thus a user may wish to select output information regarding their eating behaviour 33, output information regarding calorie consumption and categories 34, input information about the user in the settings 35; for example height, weight, sex, age.

It is thus a preferred embodiment of the present invention that activation of the application is through registration of e-mail, password and personal code, signature of “consent”) as well as input regarding gender, date of birth weight and height as well as desired weight.

Selecting the summary tab 36 would deliver output information summarizing the user's eating patterns and selection of the trends tab 37 would present output information regarding general trends in the user's eating behaviour, which may also be presented graphically by selecting the graphs tab 38.

In a further preferred embodiment of the present invention input and output processes are multiphase, thus in this embodiment the following stages are followed in the computerized system:
i. Input in the form of PUSH+PULL
ii. Data collection
iii. Analysis and output
iv. Immediate analysis
v. Daily/weekly analysis, detecting trends (causal and graphic).
vi. Messages from remote control+treating relation with central/clinical treatment system.

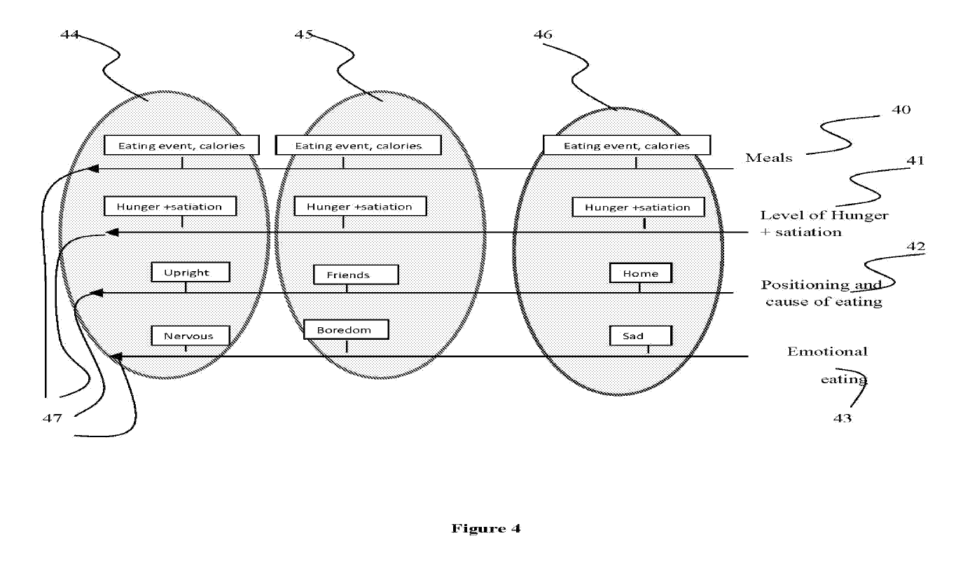

Reference is now made to FIG. 4 in the drawings, illustrating another preferred embodiment of the present invention. In this figure the factors that are involved in eating and behaviors associated with eating are presented in a schematic manner in which meals 40 are described as eating events in which calories are consumed and in which the level of hunger and satiation 41 influence the amount consumed. The position and cause for eating 42, standing upright with friends at home and the emotional mood when eating 43, for example boredom or sadness are also factors that influence eating habits. The computerized system and method accumulates data input from separate eating events 44, 45, 46 over time lines 47 for each of the different input criteria.

In another preferred embodiment the input data may relate to any behavior requiring improvement or change in order to improve and maintain wellness in the user. Thus for example when registering to use the computerized system and while in use, the data input may be concerned with food intake data, nutritional value data, satiety data, lifestyle data, emotional state or environment data, physical state or environment data, genetic data, general health data, BMI data, weight data, temporal data, geographical data, general habit data, smoking data, prescribed drug data, alcohol intake data, self medication data, non prescribed drug data, leisure activity data, hobby data, personal relationship data, fitness data, employment data and social data or any combination thereof.

It is therefore a preferred embodiment of the present invention that the reactive output of the cellular application or software generated by the computerized device instructs and educates the user in losing weight not only by rectifying calorie intake but by instilling awareness of and changing eating habits.

It is yet another preferred embodiment of the present invention that the input options at each meal or eating event include the following input options:
a. level of hunger on a scale of 1 to 5, including a verbal description
b. level of satiation on a scale of 1 to 5, including a verbal description
c. calorie estimate per meal on a scale of 1 to 5
d. nature of eating—dichotomy grading
e. Numerical rating of emotional and psychological state of user at time of eating event
f. The system automatically feeds in date and time of meal allowing for amendments in case the user feed in data retroactively.

In this embodiment the dichotomy grading is related to the level of hunger experienced by the user at the beginning of the meal in which;

A1. I am hungry

A2. I am not hungry but . . .

At this input stage user has the option of going to A1 dichotomy grading to input the level of hunger at the beginning of the meal is graded by the user according to the scale described in Table 1.

TABLE 1
A1 Dichotomy grading: HUNGER

| 5 | 4 | 3 | 2 | 1 | Grade |
|---|---|---|---|---|---|
| Famished, | Very | Hungry in | Quite | Not | Descrip- |
| restless, | hungry, | the right | hungry: “I | really | tion |
| “I will | “I wish | degree - this | would be | hungry | |
| eat | I ate | is the exact | happy to eat | | |
| anything | earlier” | time to eat | something” | | |
| now” | | | | | |

Alternatively if the user is not hungry but still eating they are directed first to the A2 dichotomy grading before being directed back to A1. The A2 dichotomy grading scale allows for a multiple option choice in which the following options maybe available in response to the question “How do you feel?”. The A2 dichotomy grading is summarized in table 2. In this embodiment the user may select more than one option regarding their emotional or psychological state prior to the eating event.

TABLE 2
A2 Dichotomy grading: EMOTIONAL/PSYCHOLOGICAL REASONS FOR EATING

| | Grade | | | | | | | | |
|---|---|---|---|---|---|---|---|---|---|
| | 1 | 2 | 3 | 4 | 5 | 6 | 7 | 8 | 9 |
| Description | tired | nervous | Social | bored | sad | alone | happy | disturbed | Other * |
| | | | Circumstances | | | | | | |

In this embodiment when the user selects to input the grade 9 *other, the computerized system allows to the user to freely input and define “*other”. In subsequent uses of the computerized system the new description of emotional state will be assigned a value that will be available as an option in this scoring scale.

In another preferred embodiment of the present invention the user inputs information at the end of the eating event to assess their physical condition at the end of the meal. The level of satiation is graded according to the scale shown in table 3:

TABLE 3
SATIATION

| 5 | 4 | 3 | 2 | 1 | Grade |
|---|---|---|---|---|---|
| Not | Quite | Satiated | Very | Terribly | Descrip- |
| satiated | satiated but I | in the | full. I | “stuffed”. | tion |
| enough. | will be happy | correct | exaggerated | Disturbing | |
| Still | to continue | amount | | feeling | |
| hungry | eating | | | | |

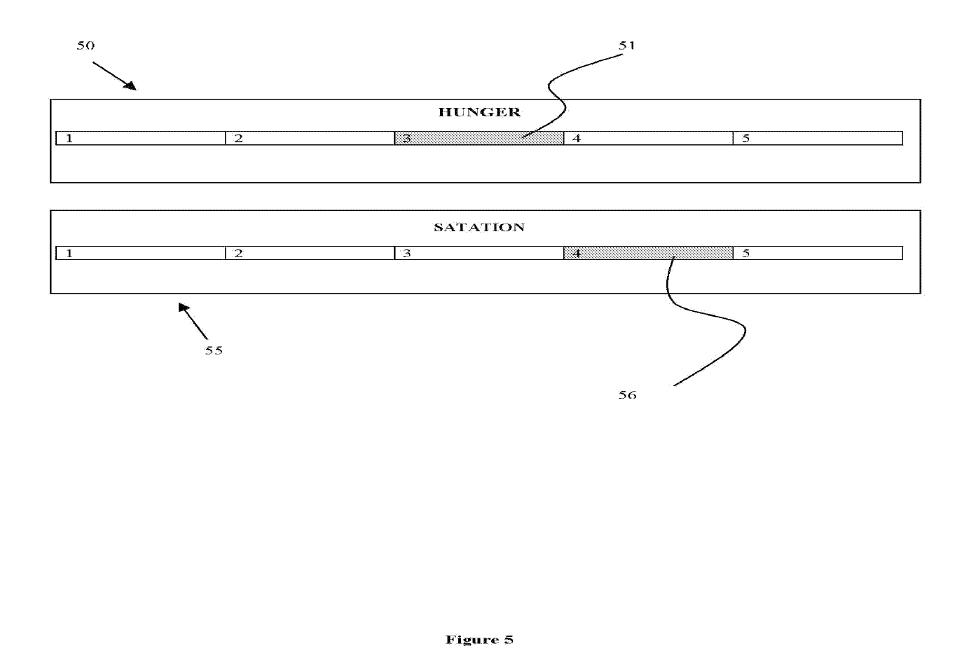

Reference is now made to FIG. 5 in the drawings, in which another preferred embodiment of the present invention is shown. In FIG. 5 a graphic visualisation of the user interface screen during the scoring process is shown. In this example both the scoring scale for hunger 50 prior to the eating event and the scoring scale for satiation 55 after a meal are shown on the same screen. In this example the user has selected a hunger level of “3” prior to the meal 51 and a satiation level of “4” after the meal 56. In such an example, the computerized weight loss system has received input that the user was “Hungry in the right degree—this is the exact time to eat” before the eating event had started and that by the end the user felt “Quite satiated but I will be happy to continue eating”. The computerized system is then able to store and process and analyze the frequency of each scoring to determine the user's eating habits and psychology. In this embodiment the natural and desired rating for the hunger scale prior to eating is 3—“hungry in the right degree—this is exactly the time to eat” and after the meal a rating of 3 on the satiation scale “satiated in the correct amount” is also desirable.

It is thus a preferred embodiment of the present invention to modify the eating habits and behavior of the user in order to achieve mostly scores of 3 for the hunger and satiation gradings.

In another preferred embodiment of the present invention, the user inputs data relating to the calorie category of the food or drinks consumed at the eating event. In this embodiment the user categorises and scores the type of meal according to its calorie content, without having to input precise calorie data as shown in table 4 below. In this scoring system the Calorie calculation refers to the average amount in each category. For example in this embodiment a hamburger meal would be categorized and scored according to its placement in the calorie category scoring system for the entire meal rather than the current method of breaking down the meal to its precise components and then allocating a calorie count for each part, in this case; the meat, bun, sauces, vegetables, chips, drink and any other part of the meal. The calorie category system enables the user to allocate a calorie category, according to the table below in a speedy way rather than the laborious method of calorie counting, thus for example the user would quickly allocate a calorie category scoring of 5 to the aforementioned hamburger meal. Research has shown that scoring calorie intake according to categories rather than precise counting of the calorie content of each item eaten in a food diary by a dietician or the dieter is comparable.

TABLE 4
EVALUATION OF AMOUNT OF CALORIES

| Grade | 1 | 2 | 3 | 4 | 5 |
|---|---|---|---|---|---|
| Approximate | Up to 200 | 200-501 | 501-800 | 801-1200 | More than |
| Calorie | calories | calories | calories | calories | 1200 calories |
| Content | | | | | |
| Description | Fruit, | Sandwich, | Regular meal; | Large meal, | Very heavy |
| | energy bar, | light meal, | coffee and | comprising | meal, several |
| | yogurt, | cornflakes | pastry, home - | heavy sauces, | courses or high |
| | coffee, | with milk, a | cooked chicken | desserts or | calorie content: |
| | slice of | small cake, | with rice, slice | several | Shwarma in |
| | bread | small portion | of pizza, sushi | courses: | pita, portion of |
| | | of ice cream, | meal, salad with | A full | humus in pita, |
| | | an iced | tuna and egg | breakfast in a | hamburger |
| | | coffee | and bread | café, a third | with chips, |
| | | | | of a baguette, | asian-style stir- |
| | | | | falafel meal, | fried noodles |
| | | | | pasta meal in | |
| | | | | a restaurant | |

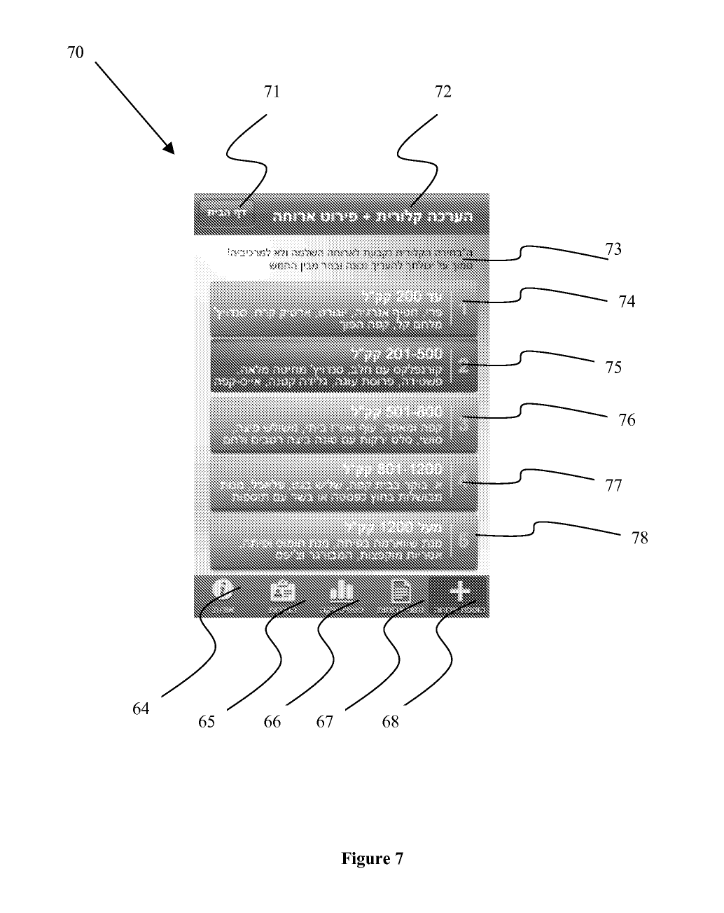

Reference is now made to FIG. 7 in the drawings in which a screen shot of the calorie category scoring in hebrew is shown 70, in this screen shot the following information and options are provided on the screen, an option to return to the home page 71, title for the page describing the page as a calorie estimation and meal description page 72, an explanation that the meal is described according to meal category and not according to its individual components 73, an option to select different calorie scores from 1 to 5 with a summary of each score, thus the user can score a 1 rating 74, 2 rating 75, 3 rating 76, 4 rating 77 and a 5 rating 78. Furthermore the screen shot offers the user general menu options including viewing or editing of the settings and personal information of a current user 65, information, help and guidance relating to the computerized system and application 64, graphs and statistical analysis 66, historic details and summaries of meals and eating events 67 and an option to input details of a new eating event 68.

In this embodiment the recommended calorie consumption for weight loss will be calculated according to Estimated Energy Requirements (EER) for each individual user based on the personal data input during the registration process. In order to obtain weight loss, the computerized system would then recommend a calorie intake equivalent to 500 calories less than the EER according to the following equation:

Recommended calorie intake: EER−500 calories

In addition a further calculation would be made to determine the minimal recommended calorie consumption for resting metabolic rate (RMR) according to the Mifflin-St Jeor equation of 1990, also based on the personal input data during the registration process.

Thus according to this embodiment the user would be provided with a recommended calorie intake range based on the two equations described above.

In a further dichotomy grading of B1 and B2 after the eating event or meal data input relating to the manner of eating is provided to the computerized system at the end of a meal, in another preferred embodiment of the present invention. In the B1 and B2 dichotomy grading the manner of eating is assessed and scored. Thus for example in the B1 dichotomy grading the physical position of the user while eating is described according to table 5 and table 6 summarizes B2 dichotomy grading in which the user describes whether they were concentrating on eating at the time of the eating event or not and the reasons for not concentrating. Also in this embodiment the speed of eating is graded on a scale of 1 to 10 (slow to fast).

TABLE 5
B1 dichotomy grading: EATING POSITION

| Grade | [symbol] | [symbol] |
|---|---|---|
| Description | Sitting down | Standing up |

TABLE 6
B2 dichotomy grading: CONCENTRATION ON THE MEAL:
In this embodiment the user describes whether they
concentrated on the meal or not, if not:

| 5 | 4 | 3 | 2 | 1 | Grade |
|---|---|---|---|---|---|
| Other (free | Reading | Watched TV | Spoke on | Ate | Descrip- |
| Record- | | | the phone | with | tion |
| multiple | | | | company | |
| choice | | | | | |
| selection. | | | | | |

In another preferred embodiment of the present invention, the computerized system collates and carries out the appropriate statistical analysis of the data input from the user and is able to determine the frequency and prevalence of the data input. For example in this embodiment the computerized system would be able to determine how often a user eats when they are not really hungry and how often they eat when they are hungry to the right degree and all possible options and combinations. The computerized system is able to determine how often the user displays different eating behaviors including all the descriptive criteria, for example how often does the user eat standing up, when they hungry at the level of 2, until they reach a satiety level of 3 at a specific hour of the day, or any other combination of scoring or data input options.

It is therefore a preferred embodiment of the present invention to provide a computerized system for controlling weight or addictive behavior my monitoring and analyzing behavior of the user in a manner that is easily accessible at all times. Further in this embodiment, the grading scales and method of inputting data and providing output is rapid and provides an immediate response.

In a further preferred embodiment of the present invention, the computerized weight loss system, provides application outputs of different types. In one embodiment the application output is a summary output—TYPE A—provided at a preset time at the end of each day. The prompt for the daily summary can optionally be a preset time set by the user and/or the first activation of the day, for example in the morning to receive the previous day's summary. It is also in this embodiment that a user can request a summary of a defined period at any point they wish.

Examples of daily summaries include:

### EXAMPLE 1

1. “Today/yesterday you ate X amount of calories”
2. “You ate X number of times over your suitable amount of satiation.”

| Output | Condition |
|---|---|
| Well done. Continue listening to your sense of | 100% hunger level = 3 |
| hunger | |
| Well done. Continue listening to your sense of | 100% satiation level = 3 |
| satiation | |

3. “You described your eating as “not hungry” X number of times”
In another preferred embodiment a second type of “Teaser” output—TYPE B—is provided by the computerized weight loss system immediately on completion of the input. In this embodiment the user is provided with a large selection of short popup teasers in direct reaction to the user's behavior. In this embodiment the “Teasers” may also include referrals and links to external information on the internet. The software of the computerized system has two types of reactive conditions to produce the “Teasers”;
1. Simple Conditions=Reaction to input of data to a certain meal
2. Complex Conditions=Behavioral pattern that repeats itself (for example, the user munches quite a lot at the same time or every Sunday, at the same time, he eats standing up.

Examples of “Teaser” output are summarized in Table 7 below:

TABLE 7
TEASER OUTPUT

| Subject | Condition | Output |
|---|---|---|
| Meal time | Additional eating after less than 30 | “Are you sure you are hungry?” |
| | minutes from previous meal | |
| | A meal delayed over 3 hours (and | Screen changes to a more and more |
| | is not the first meal of the day) | dark color as the hours pass |
| | Eating after 22:00 | Do not worry! It is not true that eating |
| | | at night is more fattening |
| | 4 hours without a meal + hunger | You are eating late therefore your |
| | level 4 + 5 | hunger level is too high |
| Calories | Level 5 | Ask yourself: Did you enjoy your |
| | | meal? |
| | | Would you repeat the meal in a similar |
| | | situation in the future |
| | 33% of the minimal daily calories | Note that amounts of calories are too |
| | (RMR) until 19:00 | low up to this hour |
| | Calorie level + similar condition + | You ate too little throughout the day |
| | satitation = 5 | therefore you achieved exaggerated/ |
| | | unpleasant feelings |
| | in more than 5 meals calorie level | “Wouldn't it be better to eat one big |
| | 1 | meal over eating a few small meals” |
| Emotional | Choice of “I'm not hungry but . . . ” | Try to change the atmosphere: Leave |
| Eating | and move to selection of “feeling” | the area that you are in/call a |
| | screen | friend/read something/take a |
| | | shower/write down what you feel/ |
| | | Chant a winning mantra for weight |
| | | loss |
| | Emotional eating + satiation < 3 | Continue eating to feel satiated |
| | Emotional eating + satiation = 3 | Well done, although you were not |
| | | driven by hunger, level of satiation is |
| | | in the correct amount |
| | Emotional eating + satiation > 3 | Try to achieve a specially pleasant |
| | | level of satiation if you were not |
| | | driven to eating by your true level of |
| | | hunger |
| Number of | Amount of meals > 10 on that day | Wouldn't you like to increase your |
| meals | | meals and decrease number of meals? |
| | Amount of meals > 2 on that day + | You are not satiated after some of the |
| | satiation < 3 | meals, increase meals |
| | Amount of meals > 2 in a certain | Note that this time of day is intended |
| | time category | for “munching” |
| Hunger & | Hunger = 1 + Satiation = 1 | You may not have interpreted |
| Satiations | | correctly sense of hunger, add food |
| | | for satiation |
| | | Wouldn't it be better to wait a while |
| | | for a clearer sense of hunger? |
| | Hunger > 3 | Wait! Drink at least 2 cups of |
| | | beverage or eat a fruit at the beginning |
| | | of the meal |
| | | Be your own manager, take |
| | | responsibility and choose correctly |
| | | Note, add intermediate meals so that |
| | | you do not reach a high level of |
| | | hunger |
| | | In the previous meal you were not |
| | | sufficiently satiated (on the condition |
| | | that your satiation level in the |
| | | previous meal was <3 |
| | Hunger > 3 + > number of times in | Recently you arrived to the meal too |
| | the last week in the same category | hungry at the X hours |
| | of time of day | |
| | Satiation = 1 | Consider adding food to achieve |
| | | proper level of satiation |
| | | Amount of calories too little, most |
| | | likely sense of hunger will be much |
| | | higher in the next couple of hours |
| | Hunger = 1 | Are you sure you want to eat now? |
| | Hunger > 3 + satiation = 3 | “Well done, you have succeeded to eat |
| | | in the right amounts despite increased |
| | | hunger” |
| | | No doubt you are the manager (!) of |
| | | this meal |
| | Satiation > 3 | Try to use a smaller plate preferably |
| | | 20 c″m wide |
| | | “Was the meal at least tasty?” |
| | | “Was this a suitable situation for |
| | | overeating?” |
| | | “Do not be ashamed- determine the |
| | | exact amount of food you are being |
| | | served” |
| | | “Concentrate on the meal: turn off the |
| | | phone and computer” |
| | | “Try to serve less on the plate and take |
| | | extras if you want” |
| | | “Drink a lot during the meal” |
| | | “Try eating a salad or soup as a first |
| | | course” |
| | | “Do not try to decrease the amount of |
| | | food in the next meal, you may eat |
| | | more later” |
| | | “Sometimes it is fun to overeat, try to |
| | | find the right situation for it” |
| | | “Not every caloric addition is |
| | | inappropriate, try looking for the right |
| | | situation for it” |
| | | “Be a manager and don't be managed” |
| | | “Being wise in retrospect- check what |
| | | is more appropriate to do next time” |
| | | “Practice over and over, your sense of |
| | | satiation” |
| | | “Don't expect miracles, practice will |
| | | bring improvements” |
| | | “Make a habit-always leave a tenth on |
| | | the plate” |
| | | “Drink at least 3 cups of water during |
| | | the meal” |
| | | “Make sure that the meal lasts longer |
| | | than 10 minutes” |
| | | “Don't despair, keep practicing” |
| | | “Is there a repeating sign indicating |
| | | non-controlled eating” |
| | | It takes a lot of practice to make |
| | | “listening to yourself” second nature. |
| | | Did you overeat because you were |
| | | afraid to feel hungry? |
| | | Note, what taste or type of food |
| | | encourages uncontrolled eating? |
| | Satiation = 2 | Consider adding food to your meal |
| | Hunger > 3 + satiation = 2 | You reach your meal too hungry and |
| | | are not satiated, you will probably eat |
| | | a lot in the next few hours |
| | Hunger > 3 + Satiation > 3 | You reached your meal too hungry |
| | | and are too satiated. Listen to your |
| | | sense of hunger and avoid unpleasant |
| | | hunger |

In a further embodiment of the present invention a third type of output is provided to the user from the computerized weight loss system of the present invention. In this embodiment the output is graphic, with summary graphs of the data input and verbal descriptions and summaries of the graphical output—TYPE C.

TYPE C output is described in the examples below:

### EXAMPLE 2

TYPE C Output for Subject—Hunger
Graph: Axis X; hunger categories (categorical), axis Y; frequency (consecutive)
Graph type: bar charts in percentages
Explanations and analysis of the graphs: you are usually hungry at rate X
*Teaser

| condition | output |
|---|---|
| Highest frequency | Well done, you are usually hungry in the |
| hunger = 3 | right amount |
| Highest frequency | Usually you arrive too hungry (connect |
| hunger > 3 | statistically with sense of satiation after |
| | the meal) |
| | Try adding intermediate meals |
| | Increase number of main meals |
| | Decrease the gap of time between the meals |
| | Pay attention to your true sense of hunger |
| Highest frequency < 3 | Note, you do not arrive to meals hungry in |
| | the right amount |

Statistical test to compare between levels of hunger in week/month X with week/month Y

### EXAMPLE 3

TYPE C Output for Subject: Satiation
Graph: Axis X; satiation categories (categorical), axis Y; frequency (consecutive)
Graph type: bar charts in percentages
Explanations of the graph: you are usually satiated at rate X

*Teasers:

| condition | output |
|---|---|
| Highest frequency | Well done, you are usually satiated in the |
| satiation = 3 | right amount |
| Highest frequency | Usually you eat over your correct level of |
| satiation > 3 | satiation (connect statistically with sense |
| | of satiation after the meal) |
| | Try drinking a lot during the meal |
| | Serve in a smaller plate and have extras |
| | as required |
| | Stop eating intentionally after 3 minutes and |
| | enjoy the feeling |
| | Pay attention to your true sense of satiation |
| Highest frequency < 3 | Note, you are not satiated enough after the |
| | meal Increase your meals |

### EXAMPLE 4

TYPE C Output for Subject: Calories
Graph; Axis X; days, axis Y; amount of calories
Graph type: Histogram diagram
Explanation of the graph: “your average calorie consumption is X”

“Teasers”

| condition | output |
|---|---|
| Calorie level < RMR | Amount of calories too low! You will not |
| | be able to keep doing this for long |
| Amount of calories | Amount of calories is suitable. You will |
| between RMR and TEE | most likely lose weight in a controlled |
| | manner |
| Amount of TEE | You will most likely keep your present |
| calories +/− 200 | weight |
| Amount of calories > TEE | You will most likely gain weight |

“T” test to compare week/month X with week/month Y

### EXAMPLE 5

TYPE C Output for Subject: Manner of Eating
Graph: Axis X—Standing up concentrated in eating, Standing up not concentrated in eating, sitting down—concentrated in eating, sitting down—not concentrated in eating, Axis Y—frequency
Graph type: bar charts or pie chart
Explanation of the graph: most of your meals are standing up/sitting down concentrated/not concentrated
“Teasers”

| Condition | Output |
|---|---|
| >25% of the meals are standing up | Eating sitting down and attentive |
| and not concentrating on the meal | is more fulfilling and decreases |
| | amount of calories |

### EXAMPLE 6

TYPE C Output for Subject: Manner of Eating
Graph: Axis X nature of eating when not concentrated—alone, talking to others, reading, watching TV, axis Y frequency
Graph Type: Bar chart or pie chart
*explanation of the graph: X is the frequent case of uncontrolled eating

### EXAMPLE 7

Optional Future Graphic Descriptions:

| Subject | Axis X | Axis Y |
|---|---|---|
| Hunger and satiation | Days/hours of the day | Most frequent levels of |
| | (categorical number) | hunger and satiation |
| Hours of the day | Hour of the day | Number of meals |
| | (categorical number) | (consecutive number) |
| | 1. 05:01-12:00 | Calories (categorical |
| | 2. 12:01-16:00 | number) |
| | 3. 16:01-19:00 | |
| | 3. 19:01-22:00 | |
| | 4. 22:01-05:00 | |
| Daily number of | Days | Number of meals |
| meals | | (consecutive) |
| Emotional eating | Type of feeling | frequency |
| Calories | Gap between meals | Calories |
| | (consecutive number) | |

Further reference is now made again to FIG. 3 in the drawings in which TABS seen on the user interface screen are graphically illustrated. In a preferred embodiment, selecting the TABS relating to summary 36 and/or Behavior 33 may generate the following output options:
“most of your meals are at X hour”
“you are eating Y meals a day”
“Most of the calories are at X hour”
Summary of the statistical connection between daily calories and number of meals/gap between meals
“the average daily calorie consumption is X”
“you usually arrive to the meal at X rate of hunger”
“you are satiated enough when you eat sitting down”

In another preferred embodiment of the present invention, selecting the Trends 37 TAB may generate the following output information and data on improvement or remission trends:
1. trends in sense of hunger before eating: categories 1-3 compared to 4+5
2. trends in sense of satiation after eating 1-3 compared to 4-5
3. amount of daily calorie in emotional eating
4. daily amount of calories
5. weight follow-up

In a further preferred embodiment of the present invention, selecting the Calories TAB 34 may generate the following output information and data;
1. Graphs: axis X; days, axis Y; calories
2. Calorie levels versus:
   a. Reason for eating: hunger/non-hunger
   b. Success in eating at the right time according to the hunger scoring and paying attention to sense of satiation versus recommended consumption of calories for weight loss which should be less than the total energy expenditure (TEE) of the user=TEE−500.

In another preferred embodiment of the present invention “PUSH” messages are automatically sent by specific prompts such as data input regarding specific behaviors of the user, including for example;
1. If it is over 4 hours from last meal or eating event—this can be neutralized at night or when the user is asleep.
2. If a full record of details was not completed in the meal, for example: forgot to mark level of satiation.
3. Prompts and allows the user to send simultaneous messages to all users such as general tips about healthy eating for example in restaurants, messages and holiday greetings

In a further preferred embodiment of the present invention, selecting the Settings TAB 35 may generate the options;
1. Changing or inserting new personal information, for example; weight, height, email address etc.
2. Change of settings for example cancellation of teaser options, language and other options.

In another preferred embodiment of the present invention the computerized system receives input through the settings option and through regular updates on eating events that allows the computerized system to adapt according to the information input provided by the user. Thus for example the system will use the personal database and behavioral data to generate output with recommendations specific to the user. Also in this embodiment the computerized system responds to the user's input history as well as the most recent input events. The computerized system is able to change its gender language from male to female and according to the language selection made by the user, for example English, Spanish, Chinese, Hebrew or any other language selected from a menu.

In a further embodiment of the present invention the output messages may be sent automatically through the computerized device database, the server or a human control system 24 hours a day.

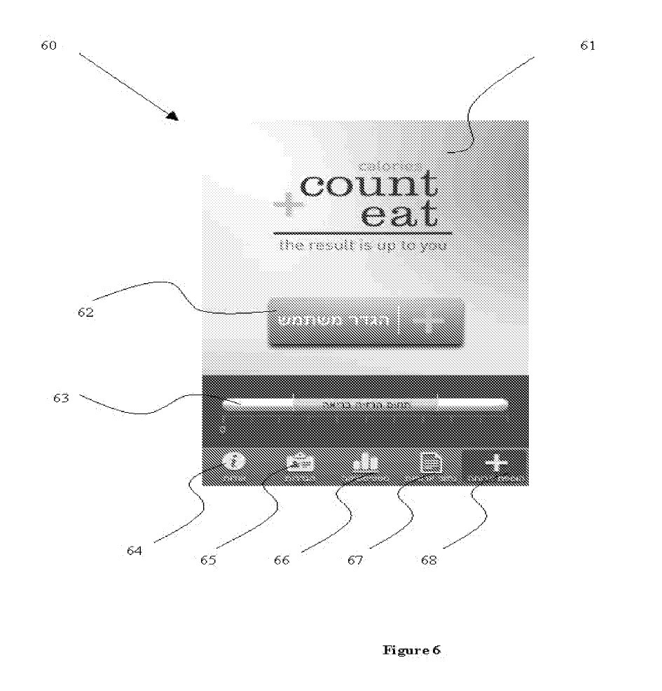

Reference is now made to FIG. 6 in the drawings, in which an illustrative example of the computerized system on a mobile phone in the Hebrew language version is shown 60. In this embodiment the screen representing the user interface of the computerized system allows a variety of possible selections. Upon entry into the computerized system a screen shot presenting the logo for the system is shown for example “count+eat” 61 as well as options for inputting data or receiving output data or historic records. Thus for example in this screen shot the user is able to select the following options; adding a new user 62, viewing or editing of the settings and personal information of a current user 65, visual representation of healthy weight loss range 63, information, help and guidance relating to the computerized system and application 64, graphs and statistical analysis 66, historic details and summaries of meals and eating events 67 and an option to input details of a new eating event 68.

It a further preferred embodiment of the present invention that the computerized system is accessible to the user through multiple devices, thus for example a user may access the computerized system through a computer user interface either through a program or the internet, or through a mobile device, PDA, digital or cable television or any combination thereof. In this embodiment a unique username and password allows the user to access the computerized system through any computerized device and especially on the internet. Also in this embodiment all data input and output options of the computerized system are available through the internet, for example historic data, summaries and trend graphs.

It is a preferred embodiment of the present invention that the computerized system contains computer programmable code adapted for calculating optimal eating behavior or optimal modifications to addictive behaviors, prompted by input by the user and the application of an algorithm able to process the input data and output an appropriate behavioral plan over time. In this embodiment, the scoring and rating scales enable rapid input of all aspects of the behavior such that the output response generated by the algorithm processing of the input provide the user with reactions and output which is holistic with regard to all behaviors, emotions and situations in which the user reports eating events or addictive behaviors. Thus the computerized device and system allow the user to self-register all associated aspects of the behavior and to receive time-resolved output generated by the algorithm that incorporates both long-term historic data and recent activities. The system therefore “learns” the behavior of the user in much the same way that a clinician such as a psychologist or psychiatrist may learn from their patient, and then generate recommendations in a holistic manner. In this embodiment the computerized system and device is able to generate output that provides recommendations and predictions to the user without the need for input from a clinician or other external human input.

In a further preferred embodiment of the present invention, the user may optionally participate or view social networks and forums associated with the computerized system through its internet website or directly through the application on a mobile network or other computerized system.

In a further preferred embodiment of the present invention the computerized system incorporates advertisements either through a search engine such as Google or in the form of paid advertising.

In a further preferred embodiment of the present invention a pocket scorecard is provided according to Table 4.

In a further preferred embodiment of the present invention a method of tracking calorie intake during a meal or food intake by scoring according to TABLE 4 is disclosed.

In a further preferred embodiment of the present invention a remote method of tracking calorie intake during a meal or food intake by scoring according to TABLE 4 is disclosed.

A trial of aspects and embodiments of the present invention was carried out as described below.

A New Method for Self-assessment of Daily Calorie Intake

The purpose of the study was to assess the validity of self-caloric evaluation, the “categories method”—a new method based on one categorical score per meal

Study Type: Observational

Study Design: Observational Model: Case-Only

Time Perspective: Cross-Sectional

Official Title: Evaluating the Validity of the “Categories” Method: a New Method for Self-report Assessment of Daily Calorie Intake

Primary Outcome Measures:

Total Calorie intake using “categories method” [Time Frame: 30-45 minutes]

Sum of every meal (the average)

Secondary Outcome Measures:

Total calories per meal using “categories method” [Time Frame: 30-45 minutes]]

Average of a category

Estimated Enrollment: 135

### Detailed Description:

Weight loss is mainly a result of reduction in daily caloric intake. Hence many people are interested in counting and tracking their daily caloric intake but In fact only 9% of people in the USA can accurately estimate the number of calories they eat (International Food Information Council Foundation, May 2011)

According to the well-known method the investigators need to know the caloric value of each and every component our dish or meal. For example: a Breakfast that contains a glass of orange juice, a small bagel, spread with cream cheese shall be calculated as: orange juice 90 kcal+bagel 192 kcal+cream cheese 98 kcal=total 380 kcal.

This method is very complicated, especially when the investigators need to calculate a dish that its ingredients are not straightforward such as: Cob salad or Shepherd's pie, Even a slice of pizza, it is very difficult to estimate the fat, carbohydrate or protein a piece of pie contains let alone the caloric volume.

The present invention provides a new method called “The categories method”: a single categorical estimation per meal based on a set of five categories:

Category 1 less than 200 kcal, Category 2 200-500 kcal, Category 3 501-800 kcal, Category 4 801-1200 kcal, Category 5 above 1200 kcal

After choosing the category the investigators use an average of the category (e.g., category 3: 500-800 Avg=650 kcal). Using the same Breakfast description as above: this meal will be considered as category No. 2 (200-500 Kcal), and chicken and rice at the restaurant will considered as a category 4 (800-1200 Kcal).

This method simplifies the process of counting calories, especially with assembled or cooked meal, it makes it easier to track any given meal even to those how are not so familiar with the caloric value of a product.

Research Protocol:

Participants were interviewed face to face on 1 occasion to obtain 24 hours recall using 4 pass method include 1. Quick review of the meals. 2. Food items that may be forgotten 3. Time and place of each meal 4. Assessment of food quantities and dimensions using a photograph published by the Israeli Ministry

This method is implemented with U.S. Department of Agriculture (USDA) as part of nutritional surveys and is used for “MBT” Survey of the Israeli Ministry of Health

Using “Tzameret” software system (food composition program of the Israeli Ministry of Health) The investigators input the 24 HR recall food list and quantity to calculate caloric values for every meal and the total calorie intake.

Using the “Categories Method” the investigators calculate the caloric value of a meal by giving the average value of each category (record in parentheses).

Reliability of the “categories method” compare with the 24 HR recall was statistically calculated.

Eligibility

Ages Eligible for Study: 21 Years to 65 Years

Genders Eligible for Study: Both

Accepts Healthy Volunteers: Yes

Sampling Method: Non-Probability Sample

Study Population

Community sample

Criteria

Inclusion Criteria:

Ages 21-65

Body mass index (BMI) between 20-45 kg/m2:

Clinical dietitians/Nutritionists

Preliminary results of the above study have shown that there is a mere 10% deviation using the categories method of daily calorie count when quantified according to calorie range categories, rather than individual foods.

1. A device for providing a subject with a behavioral plan for maintaining or improving wellness comprising a computer usable medium having a computer readable program code embodied therein, said computer usable medium comprising
a. a means of inputting data into a retrievable and updatable database, said input data concerned with food intake data, nutritional value data, satiety data, hunger data, lifestyle data, emotional state or environment data, physical state or environment data, genetic data, general health data, BMI data, weight data, temporal data, geographical data, general habit data, smoking data, prescribed drug data, alcohol intake data, self medication data, non prescribed drug data, leisure activity data, hobby data, personal relationship data, fitness data, employment data and social data or any combination thereof
b. a retrievable and updatable database comprising said data
c. a processing means comprising a computer programmable code for processing said input data to produce output data
d. a display means for displaying information concerned with said input and output data wherein said computer programmable code is adapted for calculating the optimal eating behavior to be prompted to said subject based upon application of an algorithm, said algorithm provided for processing said input data and outputting a time resolved behavioral plan

2. The device according to claim 1 wherein said behavioral plan is concerned with diet and eating.

3. A device incorporating a numerical scale for counting calorific intake which in conjunction with a computer implemented system and device is useful for weight control and wherein daily calorie count is quantified according to calorie range categories, rather than individual foods.

4. A device incorporating a numerical scale for characterization of the reason for eating—“the mental environment”, for example boredom, sadness, joy, hunger or any other emotion associated with eating wherein said device incorporates a numerical scale for characterization of the physical environment for eating, for example eating while eating alone, in company, in front of the TV or while carrying out a different activity, eating at home or in a restaurant.

5. The device according to claim 2 wherein said device is adapted to process input data consisting of a numerical rating of between 1 to 5 for hunger combined with a verbal description immediately prior to a meal or food intake further wherein said device is adapted to process input data consisting of a numerical rating of emotion combined with a verbal description immediately prior to a meal or food intake.

6. The device according to claim 2 wherein said device is adapted to process input data consisting of a rating of between 1 to 5 for satiation combined with a verbal description immediately following a meal or food intake.

7. The device according to claim 2 wherein said device is adapted to process input data consisting of a rating of between 1 to 5 for calorie categories immediately following a meal or food intake.

8. The device according to claim 2 wherein said device is adapted to process input data consisting of a visual rating of eating position during a meal or food intake.

9. The device according to claims 2 wherein said device is adapted to process input data consisting of a numerical rating of between 1 to 5 for the level of concentration on eating and physical environment during a meal or food intake.

10. The device according to claims 2, wherein A1 dichotomy grading for hunger immediately prior to a meal or food intake is scored according to TABLE 1.

11. The device according to claims 2, wherein A2 dichotomy grading for emotional and psychological reasons for eating immediately prior to a meal or food intake is scored according to TABLE 2.

12. The device according to claims 2 wherein the level of satiation immediately following a meal or food intake is scored according to TABLE 3.

13. The device according to claims 2, wherein calorie intake during a meal or food intake is scored according to TABLE 4.

14. The device according to claims 2 wherein the B1 dichotomy grading of eating position is scored according to TABLE 5.

15. The device according to claims 2, wherein the B2 dichotomy grading of eating environment and concentration during a meal is scored according to TABLE 6.

16. The device according to claim 1 wherein said behavioral plan is concerned with types of addiction or with eating disorders such as bulimia or anorexia

17. The device according to claims 1 wherein said addiction is selected from the group consisting of alcoholism, non-prescription drug addiction, prescription drug addiction, sex addiction, smoking, computer gaming addiction, gambling addiction, and any other forms of addiction.

18. The device according to claim 1 wherein said device is adapted for use in therapeutic programs or in conjunction with drug therapy or in conjunction with psychological or psychiatric therapy.

19. A system for providing a subject with a behavioral plan for maintaining or improving wellness comprising a computer usable medium having a computer readable program code embodied therein, said computer usable medium comprising
a. a means of inputting data into a retrievable and updatable database, said input data concerned with food intake data, nutritional value data, satiety data, lifestyle data, emotional state or environment data, physical state or environment data, genetic data, general health data, BMI data, weight data, temporal data, geographical data, general habit data, smoking data, prescribed drug data, alcohol intake data, self medication data, non prescribed drug data, leisure activity data, hobby data, personal relationship data, fitness data, employment data and social data or any combination thereof
b. a retrievable and updatable database comprising said data optionally residing on a server
c. a processing means comprising a computer programmable code for processing said input data to produce output data
d. a display means for displaying information concerned with said input and output data wherein said computer programmable code is adapted for calculating the optimal eating behavior to be prompted to said subject based upon application of an algorithm, said algorithm provided for processing said input data and outputting a time resolved behavioral plan.
